(19) United States
(12) Patent Application Publication
(10) Pub. No.: US 2017/0156633 A1
(43) Pub. Date: Jun. 8, 2017

(54) RESPIRATION MONITORING APPARATUS

(71) Applicants: Sheffield Hallam University, South Yorkshire (GB); Sheffield Children's NHS Foundation Trust, Sheffield (GB)

(72) Inventors: John TRAVIS, South Yorkshire (GB); Reza SAATCHI, South Yorkshire (GB); Ben HUGHES, South Yorkshire (GB); Anthony JONES, South Yorkshire (GB); Alan HOLLOWAY, South Yorkshire (GB)

(21) Appl. No.: 15/437,268

(22) Filed: Feb. 20, 2017

Related U.S. Application Data
(63) Continuation of application No. PCT/GB2015/052407, filed on Aug. 19, 2015.

(30) Foreign Application Priority Data
Aug. 20, 2014 (GB) 1414817.5

(51) Int. Cl. A61B 5/087; A61B 5/00; A61B 5/097

(52) U.S. Cl. CPC A61B 5/0878; A61B 5/097; A61B 5/725; A61B 5/746; A61B 5/0002; A61B 5/7225; A61B 5/742; A61B 5/7405; A61B 2560/0443; A61B 2503/40; A61B 2503/06

(57) ABSTRACT

Disclosed is a hand-held respiration monitoring device configured to measure the breathing pattern of a patient so as to output respiration rate based data. The device comprises a sensor to be sensitive to a respiration airflow velocity flowing through the device with an identification of the change in the velocity being filtered by high and lowpass filters. A method of respiration monitoring is also disclosed.

### FIELD OF INVENTION

The present invention relates to apparatus and method for monitoring respiration of a human or animal using a sensor to detect a change in velocity of air flowing through part of the apparatus resultant from a flow of exhaled air.

### BACKGROUND ART

Respiratory rate is an important physiological measure used in clinical and sports environments to examine the health or performance of an individual. This measurement is even more important in vulnerable patients, for example the critically ill, neonates, infants and the elderly.

Respiration monitoring can be contact or noncontact based. In contact based respiration monitoring approaches, the sensing device is attached to the subject's body. A widely used contact based respiration monitoring method relies on thermistors being placed close to nostrils to detect the temperature of exhaled and inhaled air. Another contact approach involves the use of strain-gage pressure sensors incorporated in a strap to detect chest and abdominal movements. Example electronic temperature sensing monitoring devices are described in SU 584845; US 2009/221888; US 2007/167855; GB 2039741; WO 99/34864; US 2005/096558; U.S. Pat. No. 5,081,866; U.S. Pat. No. 4,830,022; WO 2011/148159 and Plakk P et al: ‘Hot Wire Anemometer for Spirography’ Medical and Biological Engineering and Computing Springer, Heildeberg, Del., Vol 36, No 1, 1 Jan. 1998, pages 17-21.

However, conventional respiration monitoring devices are disadvantageous for a number of reasons. Principally, due to their limited sensitivity, the hand-held or patient mounted device must be positioned in very close proximity to the patient mouth or nose. Close positioning is necessary to try and reduce background noise (due to the surrounding airflow currents) and to ensure a sufficient volume of the exhaled airflow is directed over the sensor. Respiration monitoring of children is a particular problem as the positioning in close proximity of a device can have a detrimental psychological impact on the child which in turn affects their breathing rate leading to inaccurate results, incorrect diagnosis and inappropriate or ineffective treatment.

Accordingly, what is required is a respiration monitoring device that addresses the above problems and is sufficiently sensitive to obviate a need for positioning directly in front of the mouth or nose.

### SUMMARY OF THE INVENTION

It is an objective of the present invention to provide a respiration monitoring device that is configured for use with humans, animals and in particular children that is effective to provide accurate and reliable respiration rate data whilst being positioned non-obtrusively or discreetly at the patient so as to not affect their typical respiration characteristics.

The objectives are achieved by configuration of a respiration monitoring device that is very sensitive to detect respiration when positioned at a distance spaced-apart from the patient nose or mouth.

The sensitivity is achieved via a sensor mounted within an airflow tunnel that is configured to sense a change in the air flowing through the tunnel. In particular, and according to one aspect, the apparatus is configured to detect a change in the velocity of air flowing through the tunnel and via the use of highpass and lowpass frequency filters the present apparatus is configured to output a respiration signal only for respiration cycles within a predetermined frequency range. In particular, the present device or apparatus comprises a highpass filter that is configured to allow the transmission of the sensor signal at frequencies that are higher than a predetermined first frequency. Similarly, the present apparatus or device comprises a lowpass filter configured to allow the transmission of the sensor signals that are associated with the respiration velocity cycles that have a lower frequency than a predetermined second frequency. Accordingly, the present apparatus and method is optimised to be sensitive to respiration cycles by filtering background noise including environmental airflow currents and other unwanted airflow streams that do not originate from, and would otherwise ‘mask’ or interfere with, the airflow from a person or animal exhaling and breathing into the apparatus.

When implemented with a temperature sensor such as a thermistor, the sensitivity of the device may be enhanced by comprising a thermistor having a fast thermal response time which may be characterised by its ‘thermal time constant’. Advantageously, the present invention may comprise a thermistor with a thermal time constant equal to or less than 2 seconds (in substantially stationary air) mounted within an airflow tunnel.

Optimisation of the capture of the exhaled airflow without the intake of undesirable volumes of background air is achieved via the relative positioning of the sensor within the airflow tunnel of the device. In particular, to optimise sensitivity, the sensor is positioned in the region of 35 to 65% of an axial length of the tunnel from an inlet port relative to an outlet port. This is advantageous such that any background air circulating in the vicinity of the device that may be ‘drawn-into’ the airflow tunnel does not affect appreciably the respiration originating airflow stream specifically at the region of the thermistor.

In a preferred embodiment, the monitoring device comprises a funnel shaped inlet port to assist the directing or channelling of the exhaled air into the airflow tunnel towards the thermistor. Due to the relative sensitivity of the sensor, the airflow velocity within the tunnel may be optimised via a specific configuration of the inlet and outlet port.

Advantageously, a baffle or airflow filter may be provided at the outlet port to desirably inhibit or selectively adjust the amount and/or velocity of air which is allowed to exit the device and hence in turn affect the rate and flow characteristics of airflow within the tunnel.

According to a first aspect of the present invention there is provided respiration monitoring apparatus to measure a change in velocity of air flowing through part of the apparatus comprising: an airflow inlet port to allow a flow of exhaled air from a human or animal into the apparatus, and an outlet port to allow the flow of exhaled air to exit the apparatus; an airflow tunnel configured to direct the flow of exhaled air from the airflow inlet port to the outlet port; a sensor to detect a change in the air flowing from the through the tunnel from the inlet port to the outlet port and to output a signal associated with the change; characterised by: an electronic highpass filter to allow transmission of sensor signals associated with the change in the flow of air above a predetermined first frequency of the change in velocity of air; and an electronic lowpass filter to allow transmission of sensor signals associated with the change in the flow of air below a predetermined second frequency of the change in velocity of air such that the signals transmitted are within exclusively the first and second frequencies; wherein an output signal from the apparatus is provided only in response to a velocity change of the air flowing through the tunnel within a predetermined velocity range.

Preferably, the temperature sensor is a thermistor having a thermal time constant equal to or less than two seconds in substantially stationary air. Optionally, the thermal time constant is less than one second, or less than 0.5, 0.4, 0.3, 0.25 or 0.2 seconds in substantially stationary air. The present apparatus is advantageous via its response time and sensitivity to be compatible for use with a user/patient that may breathe ‘normally’ whilst achieving the desired respiration monitoring. In particular, the present apparatus is specifically patient non-contact and is capable of being positioned remotely from the patient's mouth or nose by a distance in the range 30 cm to 1.5 metres or a distance of around 30 cm, 40 cm, 50 cm, 60 cm, 70 cm, 80 cm or 1 metre. Optionally, the apparatus is configured to be sensitive to exhaled air from the nose or mouth of a patient at a separation distance between the nose or mouth and an input airflow end of the apparatus in the range 0.1 metres to 1.5 metres.

The present apparatus via the configuration of the sensor and the applied current and voltage is configured to detect small changes in relatively low velocities of air flowing through the apparatus. The present apparatus is capable of detecting and being sensitive to airflow speeds in the range 0.3 to 5 cm/sec. Optionally, the apparatus is configured to be sensitive to an airflow velocities of less than 5, 3, 2, 1, 0.75 or 0.5 cm/sec.

The present apparatus is configured via the electronic circuitry, sensor, configuration of the supplied voltage, current and/or pre-set operating temperature of the sensor (i.e., thermistor) to obviate the need for a back pressure to force the airflow through the device. that is the conventional airflow rate monitoring devices typically necessitate a mask or funnel in contact with a patient's mouth or nose to contain the exhaled air and preserve the supply pressure created by the lungs. The present apparatus is advantageous to avoid the patient having to supply a forced (high pressure) breathing pattered which is not representative of their ‘normal’ respiration.

Reference within this specification to the sensor comprising a thermal time constant or sensitivity encompasses 35 to 65% of the total response of the sensor to achieve the desired trigger signal responsive to the change in the velocity of air flowing through the device.

Preferably, the thermistor is positioned at a region in a range 35 to 65% of an axial length of the tunnel from the inlet port to the outlet port.

Preferably, the sensor may comprise anyone or a combination of the set of: a thermistor; a thermopile arrangement; a thermocouple arrangement; a heated wire an infrared based sensor.

Preferably, the first frequency is in a range 0.05 Hz to 0.1 Hz. Preferably, the second frequency is in a range 2 to 3 Hz.

Preferably, the thermistor is a negative temperature coefficient thermistor. Optionally, the thermistor is configurable to operate in a self-heating mode when supplied with current.

Optionally, the thermistor comprises a resistance in the range 1 k to 8 kΩ at 70° C. Optionally, the device comprises a maximum operating temperature of greater than or equal to 70° C.

Advantageously, a thermistor with a low thermal time constant does not require signal amplification that would also, in turn, amplify any ‘noise’ airflow within the tunnel resultant from turbulence in the air surrounding the device. The minimal thermal time constant thermistor also reduces the lag before the thermistor returns to its quiescent state between respiration airflow peaks. The preferred operational resistance and maximum allowable operating temperature of the thermistor are also advantageous to increase the sensitivity of the devise to detect respiration airflow accurately from a patient where the device is positioned outside the immediate vicinity of the nose or mouth. The maximum operating temperature is selected such that the ‘typical’ operating temperature of the thermistor is around 60° C. above ambient when the thermistor is operating in self-heating mode. The preferred resistance range avoids the need to generate and apply relatively high voltage to the thermistor.

Preferably, the monitoring device comprises an electrical circuit having a potential divider, the thermistor forming a part of the potential divider such that a change in a resistance of the thermistor provides a corresponding change in voltage at the potential divider.

Optionally, the thermistor may comprise a thermal time constant equal to or less than one second in substantially stationary air.

Optionally, the device may further comprise a data storage port to allow a data storage device to be removably attached to the respiration monitoring device. The present device may also comprise additional communication and data interface ports to allow wired or wireless connection of the device to a computer or other electronic device (PC, tablet etc). Preferably, the device comprises a communication device to provide wireless communication of respiration data from the device to a remote location. Preferably, the device may further comprise any one or a combination of the following set of: a potential divider; an analogue lowpass filter, an analogue highpass filter an amplifier; an analogue to digital converter; a CPU and memory; a graphic display; an audible buzzer; a light emitting device; a battery; a communication port. Optionally, the device may further comprise a PCB, a user interface and suitable software to process an output respiration rate data as a graphical waveform and/or numerical data.

Optionally, the respiration data may transfer in real-time or batch mode to a remote PC or VDU via wired or wireless communication. Alternatively, data may be stored at the device via suitable memory and processed at the device via a suitable processor for output at a visual display. Processing includes for example, determining the average respiration rate and its standard deviation, maximum and minimum respiration rate over a specific time interval. The on-device memory storage facility may also be configured to allow input and storage of a patient's details (including name, date of birth, time of data recording etc). This data may be entered via a PC or tablet and transmitted via a wired or wireless communication device or inputted directly at the device via an on-device keypad or touch screen. Respiration data acquired from the patient would then be stored within a patient's file on the device for output at the device or remotely via a PC or tablet.

In one embodiment, the device includes a rechargeable battery. A suitable port or docking station may be provided to interface with a recharging station. This port may also be used for data transfer to a remote PC or other storage and processing device. Furthermore, the device may comprise suitable electronics and/or software to provide an automatic power-off following a predetermined period of inactivity.

Optionally, the device may comprise a funnel provided at the airflow inlet port. Optionally, the funnel is detachably mounted in fluid communication with the main airflow tunnel that accommodates the thermistor. Optionally, the funnel may be integral with the main tunnel and/or a main body of the device. Preferably, the device is a hand-held device and comprises a housing suitable to be grasped by the hand of a user. The housing may define the airflow tunnel and the inlet and outlet ports. Optionally, the tunnel and inlet and outlet ports may be defined by separate components additional to the housing. The housing may be a single moulded component or may comprise multiple modular components interconnected via conventional interlock components such as clips, bayonet fixings, plugs, pins, resiliently deformable flanges and the like. Optionally, the hand-held device may be ‘hand gun-shaped’ and comprise a trigger to actuate the device between a respiration measurement ‘on’ mode and a non-measurement ‘off’ mode.

Preferably, the tunnel comprises a forward and a rearward end wherein an internal diameter of the tunnel is substantially uniform between the forward and rearward ends. The forward and rearward ends of the tunnel may optionally define the inlet and outlet ports respectively. Optionally, the tunnel ends may comprise baffles, filters or at least one obstruction to control and in particular to restrict the passage of air to and from the tunnel. Such arrangements may be advantageous to selectively adjust the airflow velocity within the tunnel in contact with the thermistor.

Optionally, an internal diameter of the tunnel decreases from the forward to the rearward end. Optionally, the tunnel may be funnel shaped between the forward and rearward ends. In such an embodiment, the device may not further comprise an additional funnel provided at the inlet port such that the forward end of the tunnel defines the inlet port and the rearward end of the tunnel defines the outlet port. Accordingly, the inlet port may comprise a larger cross sectional area than the outlet port.

Preferably, in a plane extending perpendicular to a longitudinal axis of the tunnel, the temperature sensor is positioned substantially centrally within the tunnel. To optimise the sensitivity of the thermistor to the respiration airflow within the tunnel, the thermistor may be mounted via its ‘bare’ leads within the tunnel. However, the thermistor may be mounted at a permanent or releasable mount within the tunnel such that the thermistor main body is located substantially centrally in a radial direction relative to the longitudinal axis extending through the tunnel. Preferably, the tunnel is substantially straight between its forward and rearward ends.

Optionally, the device may comprise a sensor cartridge detachably mounted at the device and configured to mount the thermistor to allow the thermistor to be removably mounted within the tunnel. Such an arrangement is advantageous to allow the thermistor to be removed conveniently and cleaned. Optionally, all or parts of the tunnel and the inlet and outlet ports may be detachably mounted so as to allow convenient removal for cleaning and maintenance. The present device therefore may be hygienically maintained conveniently and reliably.

Optionally, the device may comprise a sensory attraction component comprising any one or a combination of the following set of: an electronic illumination component; an electronic audible component; an electronically controlled moving component; to attract the attention of a human or animal when exhaling into the device. Optionally, the device may comprise a collar or flange mountable at the region of the inlet port configured to attract the attention of a patient to ensure they are facing the device to promote direction of the exhaled airflow into the inlet port. Such electronic components may comprise electronically powered lights, LEDs, graphic displays, buzzers, speakers, pivoting or rotating components movably mounted at the device. Such devices and components may be powered by the battery or power source that powers the thermistor and potential divider circuit.

According to a second aspect of the present invention there is provided a method of respiration monitoring to measure a change in velocity of air flowing through part of the apparatus comprising: allowing a flow of exhaled air from a human or animal into the apparatus via an airflow inlet port and allowing the flow of exhaled air to exit the apparatus via an outlet port; directing the flow of exhaled air from the airflow inlet port to the outlet port via an airflow tunnel; detecting a change in the air flowing through the tunnel from the inlet port to the outlet port via a sensor and outputting a signal associated with the change; characterised by: allowing transmission of sensor signals associated with the change in the flow of air above a predetermined first frequency of the change in velocity of air using an electronic highpass filter; allowing transmission of sensor signals associated with the change in the flow of air below a predetermined second frequency of the change in velocity of air using an electronic lowpass filter such that the signals transmitted are within exclusively the first and second frequencies; and outputting a signal from the apparatus only in response to a velocity change of the air flowing through the tunnel within a predetermined velocity range.

### BRIEF DESCRIPTION OF DRAWINGS

A specific implementation of the present invention will now be described, by way of example only, and with reference to the accompanying drawings in which:

FIG. 1 is an external perspective view of a respiration monitoring device incorporating a temperature sensing thermistor housed within an airflow tunnel according to a specific implementation of the present invention;

FIG. 2 is a cross sectional side view of the device of FIG. 1;

FIG. 3 is a partially exploded perspective view of the device of FIG. 2;

FIG. 4 is a partially exploded front perspective view of the device of FIG. 3 with selected components removed for illustrative purposes;

FIG. 5 is a partially exploded rear perspective view of the device of FIG. 3 with selected components removed for illustrative purposes;

FIG. 6 is a schematic block diagram of various electrical components forming a part of the respiration monitoring device of FIG. 1 according to a specific implementation of the present invention.

### DETAILED DESCRIPTION OF PREFERRED EMBODIMENT OF THE INVENTION

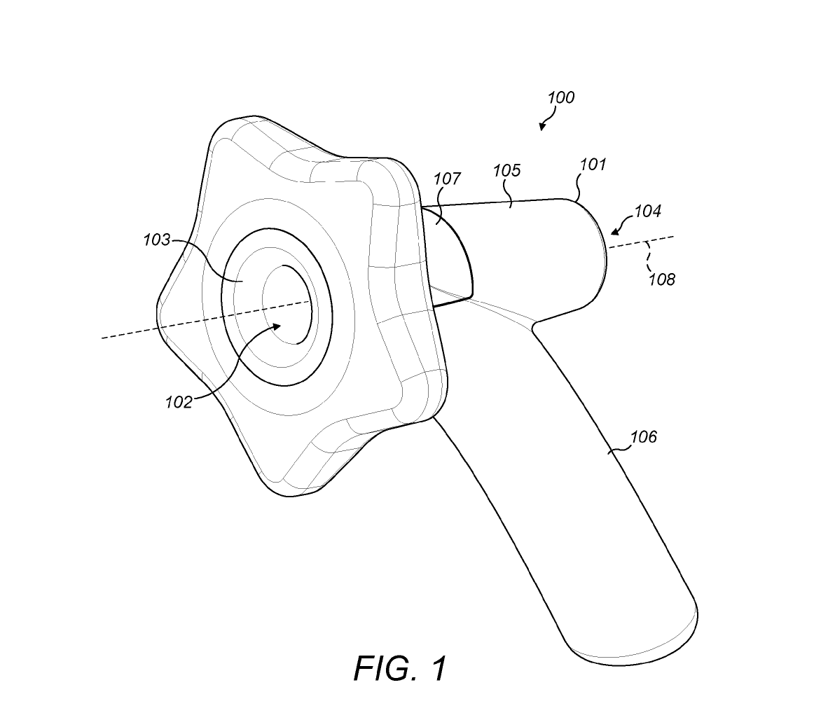

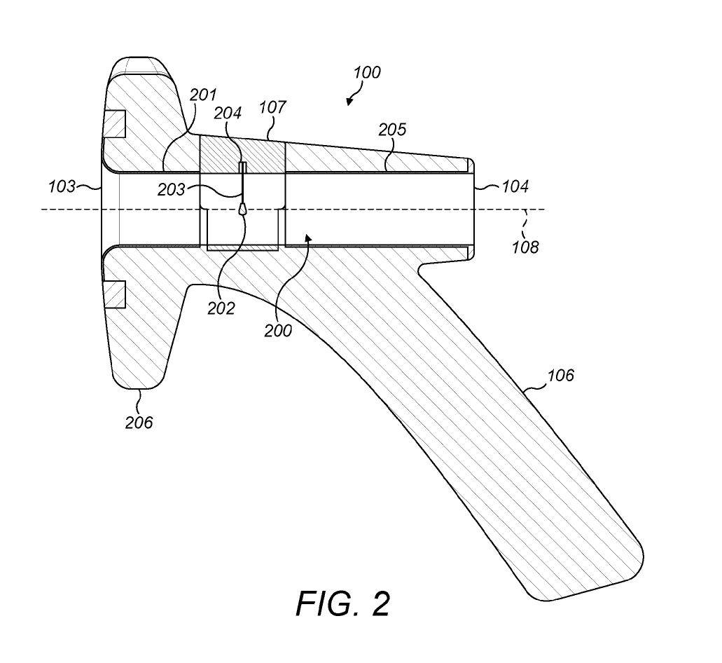

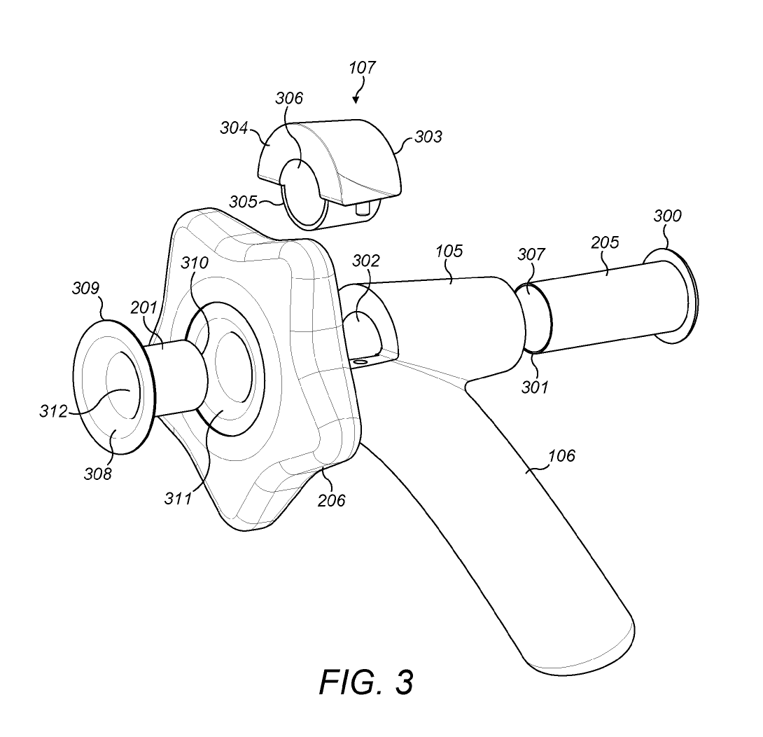

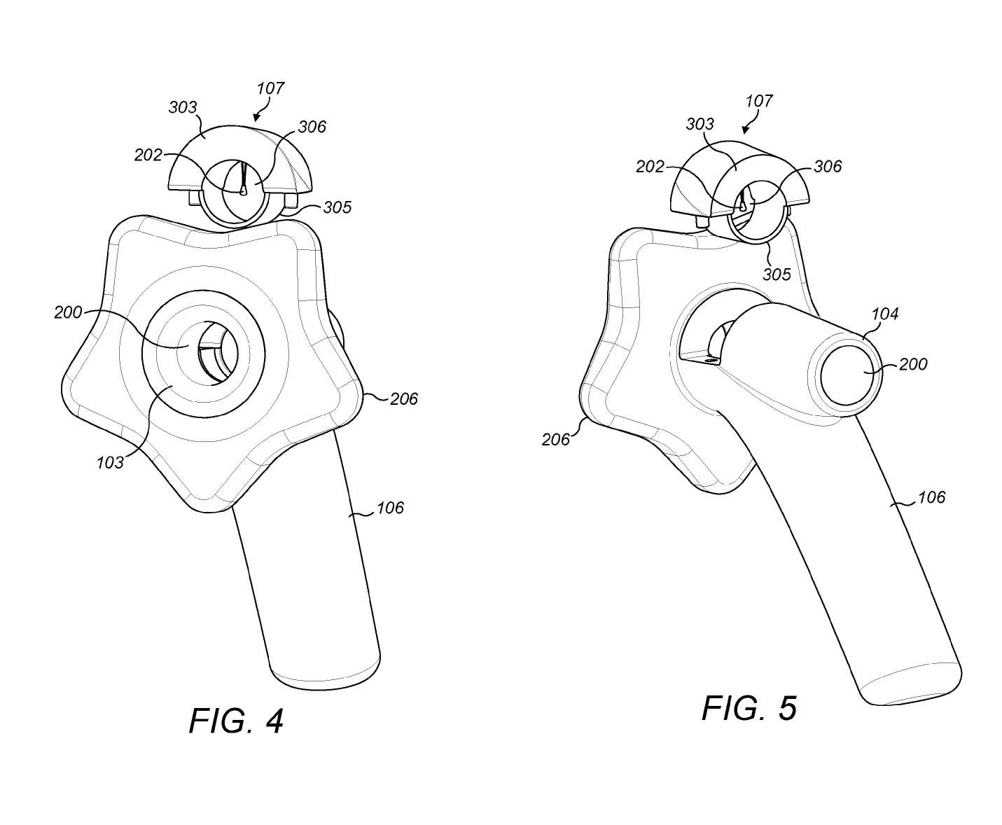

Referring to FIGS. 1 to 5 a hand-held respiration monitoring device 100 comprises a main housing 101 formed from a moulded polymer material such as a hard thermoplastic. Housing 101 comprises a hollow elongate main body 105 having a generally cylindrical configuration and centred on a central longitudinal axis 108. A handle 106 projects laterally outward from main body 105 and is formed integrally with housing 101. Main body 105 is generally hollow and defines an elongate internal airflow tunnel 200 centred on axis 108 and comprising a generally cylindrical internal facing surface. A forwardmost end of tunnel 200 defines an airflow inlet port 103 and a rearwardmost end of tunnel 200 defines an airflow outlet port 104. Tunnel 200 is configured to receive airflow resultant from the respiration of a patient breathing into an inlet region 102 of device 100. The present device 100 is configured to be hand-held and to measure respiration at a spaced-apart separation distance from the mouth or nose of a patient whilst eliminating background ‘noise’ resultant from turbulence and airflow currents surrounding device 100.

Housing 101 and in particular airflow tunnel 200 is configured to receive a substantially hollow and cylindrical inlet baffle 201 detachably mounted at the inlet port 103. Similarly, a substantially hollow cylindrical outlet baffle 205 is detachably mounted to extend along tunnel 200 from the rearward outlet port 104. Inlet baffle 201 comprises a forward inlet end 309 and a rearward outlet end 310. An airflow passage 312 is defined by baffle 201 and extends between forward and rearward ends 309, 310. Baffle inlet end 309 comprises a generally funnel or cone-shaped region 308 to assist with directing the exhaled airflow into passageway 312 to allow the exhaled air to flow through tunnel 200 within main body 105. Outlet baffle 205 similarly comprises an inlet end 301 and an outlet end 300. Inlet end 301 is configured for positioning within tunnel 200 whilst outlet end 300 is configured for positioning at outlet port 104. An elongate airflow passageway 307 extends axially between ends 301, 300 being centred on axis 108 and mountable at main body tunnel 200.

Or a device for laminating/smoothing airflow in the form of a honeycomb of round, oval or polygon shaped tubes may also be employed.

Main body 105 comprises a cut-out section 302 positioned at a substantially axial mid-region between inlet and outlet ports 103, 104. Section 302 is dimensioned and configured to receive a sensor cartridge 107 capable of being detachably mounted at main body 105. Cartridge 107 comprises a generally hollow cylindrical body 305 that defines an internal passageway 306 having an inlet end 304 and an outlet end 303. A thermistor 202 is suspended within cartridge 107 via electrical connections 203 that project radially inward from a base mount 204 located at an internal facing surface that defines passageway 306.

Thermistor 202 is accordingly suspended centrally within passageway 306 such that when cartridge 107 is mounted at main body tunnel 200, thermistor 202 is aligned on axis 108.

According to the specific implementation, thermistor 202 is positioned at a region axially towards inlet port 103 relative to outlet port 104. However, according to further specific implementations, thermistor 202 may be positioned substantially centrally in the axial direction between inlet and outlet ports 103, 104. In particular, thermistor 202 being housed within tunnel 200 is sufficiently spaced from inlet port 103 such that it is configured to be responsive to the exhaled airflow from a patient and not turbulence within the air surrounding the device 100.

Device 100 further comprises a radially extending flange 206 mounted at inlet port 103. Flange 206 comprises a central funnel shaped inlet region 311 that defines the airflow inlet portion of main body 105. Funnel region 311 is configured to mate with the cone shaped region 308 of inlet baffle 201. According to the specific implementation, the radial peripheral region of flange 206 is contoured so as to be visually appealing to a patient to facilitate obtaining and maintaining the patient's attention to ensure the exhaled airflow is directed into inlet port 103. According to further specific implementations, flange 206 may be configured with suitable illumination components and/or may be brightly coloured to assist with visual attraction. Additionally, according to further embodiments, device 100 may comprises audible components or movable components mounted at main body 105 and in particular flange 206 so as to attract and maintain the attention of a patient.

With the inlet and outlet baffles 201, 205 and cartridge 107 mounted in position as illustrated in FIG. 1, the substantially cylindrical passageways 312, 307 and 306 align axially and radially to create a continuous airflow passageway through device 100 extending between inlet port 103 and outlet port 104. According to further specific implementations, outlet baffle 205 at outlet end 300 may comprises obstructions or a filter membrane/component to at least partially restrict the outward flow of air from tunnel 200. Such an arrangement is advantageous to obtain the desired airflow velocity within tunnel 200 between inlet and outlet ports 103, 104.

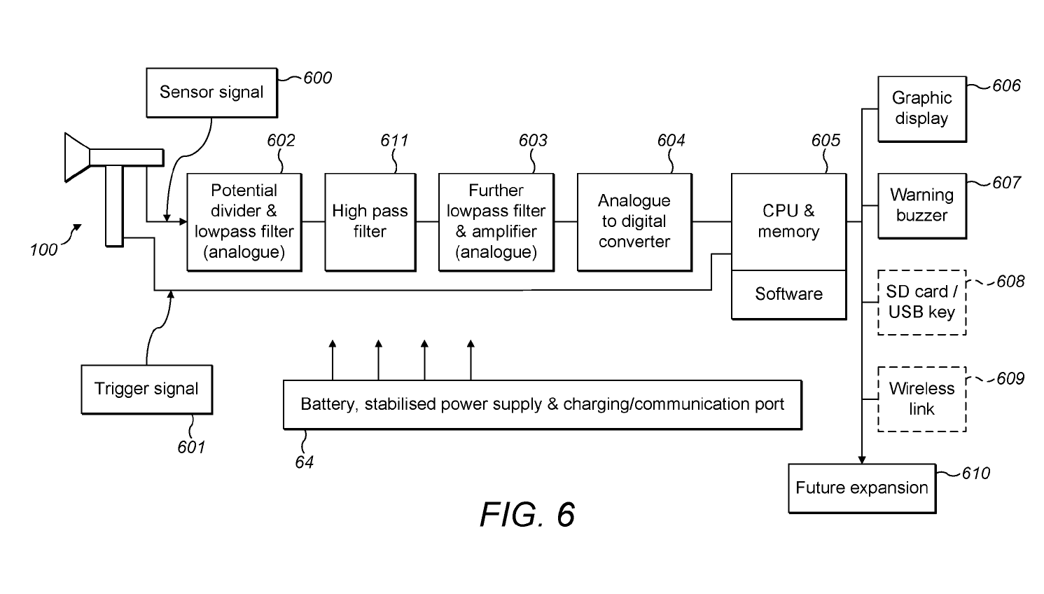

According to the specific implementation and referring to FIG. 6, device 100 comprises various electronic components. In particular, a suitable electronic circuit comprises a potential divider and analogue lowpass filter 602; a highpass filter 611; a further lowpass filter and amplifier 603; an analogue to digital converter 604; a CPU and memory supporting software 605; a graphic display 606; a warning buzzer 607; an SD card/USB port 608; a wireless communication port 609 and a suitable battery, stabilised power supply and charging port 611. The device may be further configured with expansion capability having suitable expansion ports or electronic circuitry 610 to accommodate further electrical components.

Advantageously, thermistor 202 comprises a thermal time constant of less than 3 seconds in air. According to the specific implementation, the thermal time constant is approximately equal to 1 second in stationary air and comprises a resistance of between 1 k to 8 kΩ at 70° C. to minimise the necessary operational voltage. Thermistor 202 also comprises a maximum operating temperature of between 100 to 125° C. to provide a useful working temperature of around 60° C. above ambient. A suitable thermistor for use with the subject invention comprises an MCD series resistor available from Measurement Specialists Inc., Hampton, Va. 23666, United States.

In use, the negative temperature coefficient thermistor 202 is operated in self-heating mode to detect air movement resulting from an exhaled breath flowing through tunnel 200. The output sensor signal 600 is filtered, amplified and converted to a digital signal via components 602 to 604 to be processed by CPU 605 to obtain the respiration rate. To optimise the sensitivity with regard to the typical respiration rate upper limit of around 60 breaths per minute, thermistor 202 comprises an optimised rapid time response. Thermistor 202, positioned within potential divider circuit 602, is configured such that a change in its resistance results in an appreciable change in voltage at potential divider 602.

Such an arrangement provides a relatively simple and robust configuration and minimises electrical components. In particular, thermistor 202 is placed in a lower arm of the potential divider circuit 602. On application of voltage the flow of current heats thermistor 202 being proportional to the current and thermistor resistance. The heating causes the thermistor resistance to fall until equilibrium is reached such that the heat loss by the thermistor 202 equal that generated by the internal thermistor heating. The equilibrium point may be selected to be at the thermal resistance value which could either increase or decrease the self-heating of the thermistor when cool. According to the specific implementation, sensitivity is maximised when the self-heating falls on cooling as this aids the output signal. However, there are a number of parameters which are to be satisfied and the thermistor and device electronics are selected to maximise sensitivity and suitability by optimising the following conditions: (a) an acceptable range of output voltage is available from the potential divider; (b) the power dissipated in the thermistor is self-limiting to within the specified maximum value (exceeding a rated power dissipation can destroy the thermistor); (c) the thermistor is operated as close as possible to its maximum sensitivity to cooling by air movement; (d) the power supply requirements are suited to a portable device; (e) the thermistor has an ultra-fast response of less than 1 second in substantially stationary air.

The voltage signal from the potential divider 602 is then filtered to remove extraneous noise and amplified using analogue amplifier 603. In particular, highpass filter 611, having a cut off of 0.1 Hz, is configured to allow detection of air flow variations having frequencies that are higher than this threshold and to block detection of lower frequency variations. Similarly, lowpass filter 602 and 603, having a cut off of 2 Hz, is configured to allow detection of air flow variations having frequencies lower than this threshold and to block detection of higher frequency variations. The cut off value of the lowpass filter may be increased to 3 Hz and that for the highpass filter may be reduced to 0.05 Hz.

Accordingly, the subject invention comprises a differentiating circuit to extract only variations in flow rate based on changes in the relatively low velocity of air flowing through the device.

The signal is then fed to the analogue to digital converter 604 and microprocessor 605 that provides further digital filtering and level-shifting prior to primary processing to extract restoration rate values. The restoration rate values may be time domain or frequency domain processed. Extraction via time domain involves signal peaks that are simply counted for a specific time period and the value then normalised to 1 to provide a respiration rate. This processing emulates a conventional manual technique. In frequency domain extraction, a Fast Fourier transform is performed to extract the domain frequency. The subject invention is compatible for use with either and both extraction methods with time domain offering a fast technique capable of recording irregular breathing but being potentially susceptible to noise. Frequency domain processing is less susceptible to noise when used with the subject invention but does require samples to be taken for a longer time period and may be less optimised when used with irregular respiration. The subject invention is compatible for use to obtain respiration data via both signal processing techniques such that CPU, memory, and software 605 may provide automatic correction in the event of dissimilarity between the respiration rate generated from each processing technique.

Via the graphic display 606, the device is configured to output graphically the sensor signal to an operator to optimise the position of the various components and/or the operating status of such components including optionally thermistor 202, inlet baffle 201 and outlet baffle 205. That is, being patient dependant, slightly different configurations of inlet baffle 201 may be attached to the inlet port 103 having a larger funnel geometry in an attempt to try and capture more of the exhaled breath. Additionally, an alternative cartridge 107 may comprise a thermistor 202 positioned at a different axial position relative to ends 304, 303. Outlet baffle 205 may similarly be selected from an available set of baffles with each baffle optionally having a different configuration of internal diameter and/or filter or restriction at the outlet end 300. Once the desired sensor signal is obtained, an operator presses a trigger (not shown) provided at handle 106 to start a respiration rate measurement. The trigger signal 601 is received by the CPU 605 to initiate respiration monitoring and the acquisition of data via components 602, 603, 604.

According to further embodiment, the electronic interface as detailed in FIGS. 1 to 6 incorporating thermistor sensor 202 and the associated circuitry may be adapted to suit other flow sensors employing a temperature change principle such as those based on a hot wire or those using a separate heating element in conjunction with a temperature sensor such as a thermistor, a temperature sensitive resistor, a semiconductor junction device, a thermocouple or thermopile (Seebeck effect), an infra-red device, or a microelectrical-mechanical MEMS sensor of the type D6F-P, available from Omron (www.omron.com).

According to further embodiments and as will be appreciated, the circuitry of alternate embodiments incorporating a hot wire, semiconductor junction or temperature sensitive resistor would correspond or be the same configuration as that for the thermistor sensor embodiment of FIGS. 1 to 6 without or with feedback control to maintain constant sensor temperature. As will be appreciated, circuitry for a thermocouple embodiments would employ a cold junction and a high gain amplifier.

According to further embodiments the present apparatus comprises signal processing software to detect and indicate the presence of an acceptable quality of respiratory signal.

Such an indication may be provided visibly, audibly or by touch (e.g. vibration) and, during use, may also be used to halt the data collection and processing until an acceptable signal is regained.

According to yet further embodiments the present apparatus may comprises artificial intelligence in which supplementary ports are provided to accept inputs from other physiological sensors (e.g. heart rate, body temperature, oxygen saturation) the data from which would be processed via the apparatus to provide additional health information.

According to yet further embodiments and to enhance the accuracy of the data output, the present apparatus may comprises at least two sensors (i.e., thermistors) positioned adjacent laterally with a centre septum to divide the sensors such that the sensors are located in the centre of two air flow tunnels. The signals from each sensor will be separately amplified, filtered and converted to a digital format as detailed above. However, two different data processing methods will be used to obtain respiratory rate values. Intelligent software is employed to advise and select likely values where there is disparity between the two values.

1. A respiration monitoring apparatus to measure a change in velocity of air flowing through part of the apparatus comprising:
an airflow inlet port to allow a flow of exhaled air from a human or animal into the apparatus, and an outlet port to allow the flow of exhaled air to exit the apparatus;
an airflow tunnel configured to direct the flow of exhaled air from the airflow inlet port to the outlet port;
a sensor to detect a change in the air flowing from the through the tunnel from the inlet port to the outlet port and to output a signal associated with the change;
an electronic highpass filter to allow transmission of sensor signals associated with the change in the flow of air above a predetermined first frequency of the change in velocity of air;
and an electronic lowpass filter to allow transmission of sensor signals associated with the change in the flow of air below a predetermined second frequency of the change in velocity of air such that the signals transmitted are within exclusively the first and second frequencies;
wherein an output signal from the apparatus is provided only in response to a velocity change of the air flowing through the tunnel within a predetermined velocity range.

2. The apparatus as claimed in claim 1 wherein the sensor comprises a response time equal to or less than three seconds, alternatively equal to or less than one second.

3. (canceled)

4. The apparatus as claimed in claim 1 wherein the sensor comprises any one or a combination of the set of:
a thermistor;
a thermopile arrangement;
a thermocouple arrangement;
a heated wire;
an infrared based sensor.

5. The apparatus as claimed in claim 1 wherein: (i) the first frequency is in a range 0.05 Hz to 0.1 Hz; (ii) the second frequency is in a range 2 to 3 Hz; or (iii) both (i) and (ii).

6. (canceled)

7. The apparatus as claimed in claim 1 wherein the sensor is a thermistor having a thermal time constant equal to or less than three seconds in substantially stationary air,

8. The apparatus as claimed in claim 1 wherein the sensor is positioned at a region in a range 35 to 65% of an axial length of the tunnel from the inlet port to the outlet port.

9. The apparatus as claimed in claim 1 wherein the sensor comprises a thermistor, the thermistor being a negative temperature coefficient thermistor.

10. The apparatus as claimed in claim 1 wherein the sensor comprises a thermistor, the thermistor being configurable to operate in a self-heating mode when supplied with current.

11. The apparatus as claimed in claim 1 wherein the sensor comprises a thermistor, the thermistor comprising a resistance in the range 1 k to 8 kΩ at 70° C.

12. The apparatus as claimed in claim 1 comprising a maximum operating temperature of greater than or equal to 80° C.

13. The apparatus as claimed in claim 1 further comprising a potential divider and wherein the sensor comprises a thermistor, the thermistor forming a part of the potential divider such that a change in a resistance of the thermistor provides a corresponding change in voltage at the potential divider.

14. The apparatus as claimed in claim 1 wherein the sensor comprises a thermistor, the thermistor comprising a thermal time constant equal to or less than one second in substantially stationary air.

15. The apparatus as claimed in claim 1 further comprising a data storage port to allow a data storage device to be removably attached to the respiration monitoring device.

16. The apparatus as claimed in claim 1 further comprising a communication device to provide wireless communication of respiration data from the device to a remote location.

17. The apparatus as claimed in claim 1 further comprising any one or a combination of the following set of:
a potential divider;
a lowpass filter and amplifier;
an analogue to digital converter;
a CPU and memory;
a graphic display;
an audible buzzer;
a light emitting device;
a battery;
a communications port.

18. The apparatus as claimed in claim 1 further comprising a funnel provided at the airflow inlet port.

19. The apparatus as claimed in claim 18 wherein the funnel is detachably mounted at the tunnel.

20. The apparatus as claimed in claim 1 wherein the tunnel comprises a forward and a rearward end wherein an internal diameter of the tunnel is substantially uniform between the forward and rearward ends.

21. The apparatus as claimed in claim 1 wherein the tunnel comprises a forward and a rearward end wherein an internal diameter of the tunnel decreases from the forward to the rearward end.

22. The apparatus as claimed in claim 21 wherein the tunnel is substantially funnel shaped between the forward and rearward ends.

23. The apparatus as claimed in claim 1 wherein in a plane extending perpendicular to a longitudinal axis of the tunnel, the temperature sensor is positioned substantially centrally within the tunnel.

24. The apparatus as claimed in claim 1 further comprising a sensor cartridge detachably mounted at the device and wherein the sensor comprises a thermistor, the sensor cartridge configured to mount the thermistor to allow the thermistor to be removably mounted within the tunnel.

25. The apparatus as claimed in claim 1 further comprising a sensory attraction component comprising any one or a combination of the following set of:
an electronic illumination component;
an electronic audible component;
an electronically controlled moving component;
to attract the attention of a human or animal when exhaling into the device.

26. The apparatus as claimed in claim 1 further comprising a baffle provided at the outlet to restrict the flow of air out of the outlet and the device.

27. The apparatus as claimed in claim 26 wherein the baffle is removably mounted at the device.

28. A method of respiration monitoring to measure a change in velocity of air flowing through part of the apparatus comprising:
allowing a flow of exhaled air from a human or animal into the apparatus via an airflow inlet port and allowing the flow of exhaled air to exit the apparatus via an outlet port;
directing the flow of exhaled air from the airflow inlet port to the outlet port via an airflow tunnel;
detecting a change in the air flowing through the tunnel from the inlet port to the outlet port via a sensor and outputting a signal associated with the change;
allowing transmission of sensor signals associated with the change in the flow of air above a predetermined first frequency of the change in velocity of air using an electronic highpass filter;
allowing transmission of sensor signals associated with the change in the flow of air below a predetermined second frequency of the change in velocity of air using an electronic lowpass filter such that the signals transmitted are within exclusively the first and second frequencies; and
outputting a signal from the apparatus only in response to a velocity change of the air flowing through the tunnel within a predetermined velocity range.
